(19) United States
(12) Patent Application Publication
(10) Pub. No.: US 2024/0242799 A1
(43) Pub. Date: Jul. 18, 2024

(54) METHOD AND SYSTEM FOR AGGREGATING DATA

(71) Applicant: Ciitizen, LLC, San Francisco, CA (US)

(72) Inventors: Peeyush RAI, Palo Alto, CA (US); Brian CARLSEN, Palo Alto, CA (US); Viraj NILAKH, Palo Alto, CA (US); Lisandra WEST-ODELL, Palo Alto, CA (US)

(73) Assignee: Ciitizen, LLC, San Francisco, CA (US)

(21) Appl. No.: 18/562,191

(22) PCT Filed: May 17, 2022
(86) PCT No.: PCT/US2022/029686

§ 371 Date: Nov. 17, 2023

Related U.S. Application Data
(60) Provisional application No. 63/189,962, filed on May 18, 2021.

(51) Int. Cl. G16H 10/60; G06F 40/205; G16H 50/20

(52) U.S. Cl. CPC G16H 10/60; G06F 40/205; G16H 50/20

(57) ABSTRACT

This invention relates generally to the medical technology field, and more specifically to a new and useful method for aggregating data.

### TECHNICAL FIELD

Aspects of this invention relate generally to the medical technology field, and more specifically to a new and useful method for aggregating data.

### BACKGROUND

Generally, when a patient receives treatment from medical providers (e.g., different medical professionals for different ailments), the treatment is documented in different medical data sources, (e.g., medications, specialty care, emergency room, radiology, labs, surgery, outpatient care, primary care physician (PCP), other tests, etc.). Additionally, medical providers do not have a standardized way of describing patients within the different medical sources. Thus, there is a need in the healthcare field to aggregate data from different data sources and from different providers into a single data source for a patient.

### SUMMARY

Advancements in data aggregation improve the analysis of patient data. The solutions presented herein present methods and systems for aggregating data from multiple data sources to form a complete patient profile. Advantages include, but are not limited to, deduplication of patient records, being able to track a patient's history over a period of time, and verification of diagnoses.

Some aspects of the invention include receiving a data source relating to a patient, the data source containing a data set and matching data in the data set to at least one of a set of predetermined attributes and a set of predetermined properties to produce a set of extracted attributes and a set of observed properties for the patient. In some aspects, each extracted attribute is a predetermined attribute that matches first data in the data set, and each observed property is a predetermined property that matches second data in the data set; for at least one attribute in the set of extracted attributes, identifying a set of ontologies that maps to the at least one attribute. In some aspects, a set of respective inferred properties for the at least one attribute based on the set of ontologies are determined and merging, according to a set of merging rules associated with the set of ontologies, the set of extracted attributes, the set of observed properties, and the set of respective inferred properties into a set of database records corresponding to a profile of the patient. In some aspects, database records are linked corresponding to a plurality of extracted attributes, observed properties, and inferred properties within the profile of the patient based on the set of ontologies.

In some aspects, based on the set of observed properties and the set of respective inferred properties, the set of extracted attributes are verified.

In some aspects, the merging comprises: determining that multiple attributes in the set of extracted attributes correspond to different versions of a same information; and consolidating the multiple attributes to a single attribute within the set of extracted attributes.

In some aspects, a first attribute in the multiple attributes has a first observed property, a second attribute in the multiple attributes has a second observed property, and the single attribute has both the first observed property and the second observed property.

In some aspects, the first observed property is a start date and the second observed property is an end date.

In some aspects, repeating the determining and consolidating for each group of multiple attributes in the set of extracted attributes to generate a set of single attributes within the set of extracted attributes.

In some aspects, each single attribute within the set of extracted attributes is associated with at least one date, the merging further comprising: displaying, in a user interface generated from the profile of the patient, each single attribute within the set of extracted attributes along a timeline based on its respective at least one date.

In some aspects, each extracted attribute in the set of extracted attributes is related to a respective subset of observed properties in the set of observed properties for the patient.

In some aspects, the merging comprises displaying, in a user interface generated from the profile of the patient, each extracted attribute together with its respective subset of observed properties.

In some aspects, each extracted attribute in the set of extracted attributes is related to a respective subset of inferred properties in the set of inferred properties for the patient.

In some aspects, the matching comprises: parsing the data in the data set into a set of medical terms using natural language processing; and matching each medical term in the set of medical terms to the at least one of the set of predetermined attributes and the set of predetermined properties.

In some aspects, the matching comprises: determining word embeddings from the data in the data set; and comparing the word embeddings against the at least one of the set of predetermined attributes and the set of predetermined properties to produce the set of extracted attributes and the set of observed properties for the patient.

In some aspects, the comparing comprises fuzzy matching.

In some aspects, the matching comprises: extracting statements from the data in the data set using a model trained to extract statements associated with a given attribute type; and adding the extracted statements to at least one of the set of extracted attributes and the set of observed properties for the patient.

In some aspects, the set of predetermined attributes comprises a set of predetermined attribute types, and wherein the matching comprises: extracting statements from the data in the data set using a learning model trained to identify medical terms; classifying each extracted statement as one of the predetermined set of attribute types; and adding the classified statement as an attribute in the set of extracted attributes for the patient.

In some aspects, the set of extracted attributes is an existing set of extracted attributes and the set of observed properties is an existing set of observed properties, the method further comprising: receiving a new data source relating to the patient, the new data source containing a new data set; matching the new data set to the set of predetermined attributes to produce a new set of extracted attributes and a new set of observed properties for the patient; determining that a new attribute in the new set of extracted attributes is the same as an existing attribute in the existing set of extracted attributes; and merging any new observed properties corresponding to the new attribute into the set of database records corresponding to the profile of the patient for the existing attribute.

In some aspects, identifying a set of ontologies comprises: determining a standardized code corresponding to the at least one attribute; sending the standardized code to an ontology lookup service; and returning all ontologies in a datastore associated with the ontology lookup service that are tagged as related to the standardized code.

In some aspects, repeating the receiving, matching, identifying, querying, and merging for at least one other patient.

Further features of the present disclosure, as well as the structure and operation of various aspects, are described in detail below with reference to the accompanying drawings. It is noted that the present disclosure is not limited to the specific aspects described herein. Such aspects are presented herein for illustrative purposes only. Additional aspects will be apparent to persons skilled in the relevant art(s) based on the teachings contained herein.

### BRIEF DESCRIPTION OF THE FIGURES

FIG. 1 is a flowchart depicting a method for aggregating data according to an aspect of the present invention.

FIG. 2 is a flowchart depicting a system for aggregating data according to an aspect of the present invention.

FIG. 3 is an example of a method for aggregating data according to an aspect of the present invention.

FIG. 4 is another example of a method of aggregating data according to an aspect of the present invention.

FIG. 5 is an example computer system useful for implementing various aspects.

In the following description, the same or similar elements are labeled with the same or similar reference numbers.

### DESCRIPTION OF THE PREFERRED EMBODIMENTS

Aspects of the invention will now be described more fully hereinafter with reference to the accompanying drawings, in which aspects of the disclosure are shown. The aspects may, however, be embodied in many different forms and should not be construed as limited to the aspects set forth herein. Rather, these aspects are provided so that this disclosure will be thorough and complete, and will fully convey the scope of the disclosure to those skilled in the art.

Various aspects will now be described more fully hereinafter with reference to the accompanying drawings. However, they may be embodied in different forms and should not be construed as limited to the aspects set forth herein. Rather, these aspects are provided so that this disclosure will be thorough and complete, and will fully convey the scope of the disclosure to those skilled in the art.

### 1. Overview

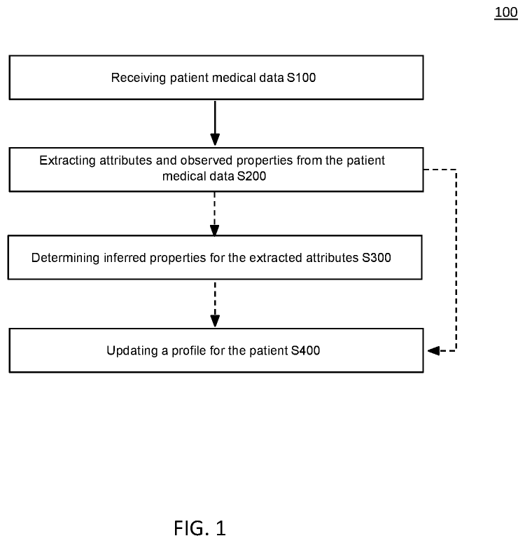

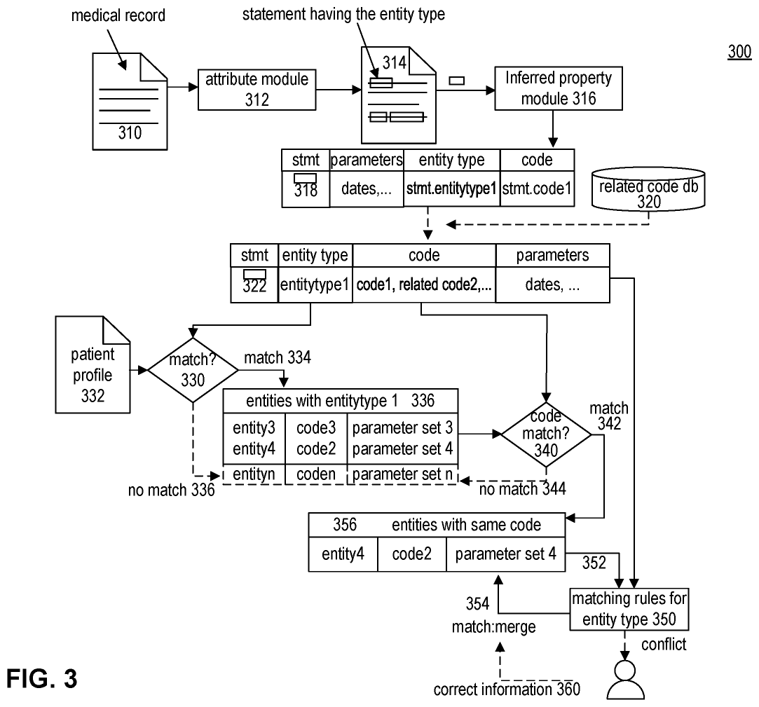

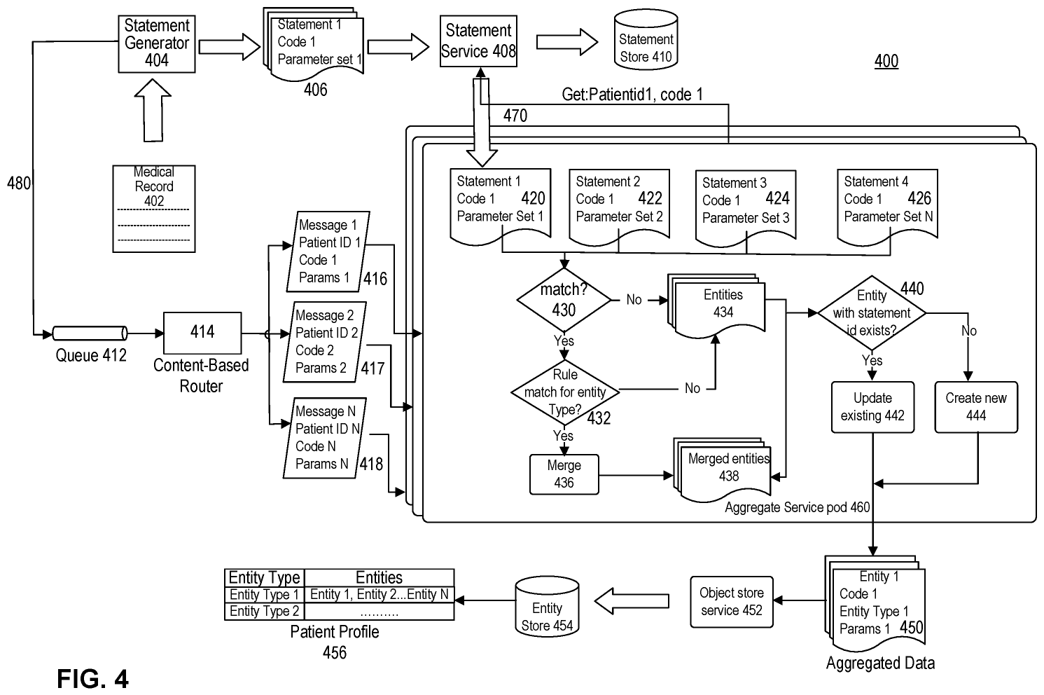

As shown in FIG. 1, the method 100 for data aggregation begins with S100, in which patient medical data is received. In S200, attributes and observed properties are extracted from the patient medical data. In S300, which is optional, inferred properties for the extracted attributes are determined. In S400, a profile for the patient is updated. The method 100 may additionally include any other suitable elements. The method 100 is further described in the context of FIGS. 3 and 4 below.

### 2. Examples

In a first example, the method and system may include receiving different healthcare documents for the patient (e.g., from the same or different medical providers); extracting attributes and observed properties from each of the different healthcare documents; determining inferred properties, such as an ontology code for each extracted attribute; and updating a profile for the patient by: adding the extracted attributes to the set of existing attributes, filtering all attributes to determine a merged set of attributes, and/or verifying conflicted attributes. This example is further described in the context of FIGS. 3 and 4 below.

### 3. Benefits

The method may confer several benefits over conventional systems.

First, in variants, the system and method processes multiple healthcare documents from the same or different providers to remove duplicate mentions of the same attribute (e.g., diagnosis, medication, procedure, etc.). De-duplication may be performed by matching the attribute, matching one or more properties, or entity, of the attribute (e.g., dates, medical codes, ontology codes), and based on the match(es), determining that the attributes are different versions of the same information and consolidating the information into a single attribute (e.g., adding properties of one attribute to the other, not adding properties, removing an occurrence of a duplicated attribute, etc.).

Within a typical healthcare document, various sections exist, such as a diagnosis section, medication section, family history section, etc. Each section of the healthcare document may be processed to extract attributes, such as a particular diagnosis, medication, or procedure, and properties, such as dates, medical codes, ontology codes, etc.

In accordance with aspects of the present invention, the healthcare documents are normalized for future use. For example, in some documents, the patient's weight, which is an attribute (also referred to herein as an entity), may be described in either kilograms or pounds. For normalization, the weight description may be altered to use the same unit throughout the patient profile analysis. If two different values for the same attribute occur, for the weight for example, then a conflict may be created, which is not desirable. Normalizing the attributes may ensure that any conflict for an attribute either does not occur or may be mitigated.

Second, the system and method may track timelines of attributes by merging attributes and properties of the attribute (e.g., dates, medical codes, ontology codes, etc.). For example, an attribute detected in a first document may be associated with a start date. The attribute may also be detected in a second document, but associated with an end date. In this example, the system and method may merge the attribute detections into a single attribute with both a start and end date. Third, the system and method may verify accuracy of attributes and properties (e.g., correct incorrectly identified attributes and/or properties, such as using new patient medical data, using historical patient medical data, using medical data from different providers to determine a consensus, etc.). For example, a first document may specify a first diagnosis for a patient, then after a few more tests, a second document may specify a second diagnosis for the patient that is meant to correct the first diagnosis (e.g., the patient is diagnosed with breast cancer, and then after a biopsy, the patient is re-diagnosed with a lump that is not breast cancer, so the not breast cancer diagnosis would replace the breast cancer diagnosis). The second diagnosis may be detected as a replacement for the first diagnosis based on ontology codes, dates, medical provider notes (e.g., included in the document), treatment plans, and/or otherwise detected.

However, the method and system may confer any other suitable benefits.

### 4. System

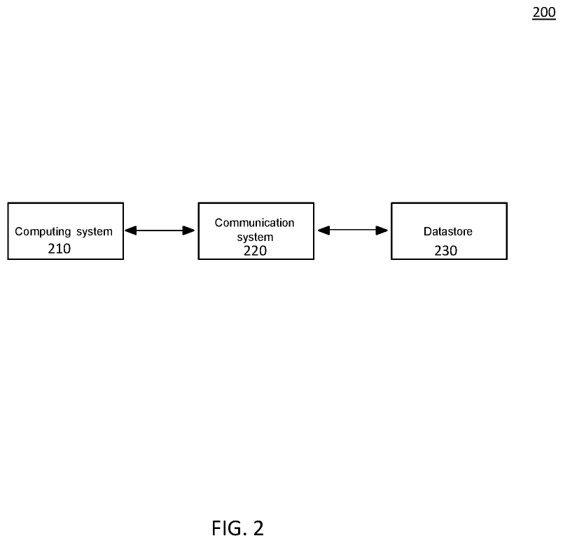

In some aspects, the method is performed using the system, as shown in FIG. 2, including: a computing system 210, a communication system 220, a datastore 230, and/or any other suitable components.

The computing system 210 may function to perform all (or a subset thereof) of the method. The computing system 210 may include a remote computing system (e.g. one or more servers), user device (e.g., smartphone, laptop, desktop, etc.), and/or any other computing system. In some embodiments, the computing system 210 may include a remote computing system and a user device that interfaces with the remote computing system via an API. In some embodiments, the computing system 210 may include a remote computing system that interfaces with a third-party service via an API. The computing system 210 may include one or more modules. However, the computing system 210 may be otherwise configured.

The communication system 220 functions to communicate data (e.g., patient medical data) from one or more third-party services to the computing system 210, optionally communicate data (e.g., profile for a patient, attributes for a patient, properties for an attribute, etc.) from the computing system 210 to the one or more third-party services, and/or perform any other functionality. The communication system 220 may include any suitable communication system, such as wired connections, wireless connections, or a combination of wired and wireless connections. In an example embodiment, one or more portions of the communication system 220 may be an ad hoc network, an intranet, an extranet, a virtual private network (VPN), a local area network (LAN), a wireless LAN (WLAN), a wide area network (WAN), a wireless wide area network (WWAN), a metropolitan area network (MAN), a portion of the Internet, a portion of the Public Switched Telephone Network (PSTN), a cellular telephone network, a wireless network, a WiFi network, a WiMax network, APIs, a Bluetooth system, any other type of network, or a combination of two or more such networks. However, the communication system 220 may be otherwise configured.

The datastore 230 functions to store data that may be used to perform the method (e.g., patient medical data, predetermined attributes, predetermined observed properties, etc.), information determined by the method (e.g., extracted attributes, merged attributes, observed properties, inferred properties, merged properties, etc.), and/or any other suitable information. The predetermined attributes and/or predetermined properties may be compared to extracted terms from patient medical data to classify extracted terms as attributes, properties, additional text (e.g., for context), provider notes, or non-relevant terms. The datastore 230 may reside in a cloud-computing environment, or it may be located on a single physical or virtual machine. The datastore 230 is generic (e.g., used across multiple patients). However, the datastore 230 may be otherwise configured.

### 5. Method

Returning to FIG. 1 as described above, a method for data aggregation according to aspects of the invention may function to: aggregate data extracted from one or more different sources of patient medical data, that may be from multiple different medical providers; update an existing patient profile with new extracted data; create a new patient profile for a new patient; and/or provide any other suitable functionality.

In some aspects, the method is performed per patient, but may additionally or alternatively be performed for multiple patients in parallel (e.g., different instances of the method may be performed for different patients in parallel), and/or the method may be otherwise performed.

In some aspects, the method is performed when patient medical data is available, such as for a single data element (e.g., document, or other record) at a time (e.g., wherein the patient medical data may be received from a third-party service when new data enters their system, periodically received from the third-party service in response to a query, received from the datastore when a new entry is added, periodically received from the datastore in response to a query, etc.), but may additionally or alternatively be performed one data element at a time for a batch of data elements, wherein the data extracted from each data element may be aggregated as a batch in S400, and/or the method may be otherwise performed.

The method is preferably performed by the system disclosed above, but may be otherwise performed.

### 5.1 S100—Receiving Patient Medical Data.

In S100, patient medical data is received. Patient medical data may be received via a data source relating to a patient, the data source containing a data set. For example, S100 may function to receive one or more data elements (e.g., document; data structure element, such as generated from an online form; or other suitable record) that may be used by the method to update a profile for the patient (e.g., in S400). The patient medical data may be received from: electronic health records, electronic medical records, physical records, consumer products (e.g., wearable monitoring devices, such as an Apple Watch™ or FitBit™), and/or other sources. Patient medical data may include: raw text, medical standard terms (e.g., medical terms in the healthcare record translated to standard terms), and/or any other suitable text. Patient medical data may include: patient records, patient supplied information (e.g., name, date of birth, height, weight, medical history, etc.), and/or any other suitable data. Examples of patient medical data include: medications, specialty care, emergency room, radiology, labs, surgery, outpatient care, PCP, other tests, and/or any other suitable data.

In a first variant, determining patient medical data may include retrieving the patient's medical data from a medical database, from a third-party database, and/or from any other suitable database. The patient medical data may be retrieved by: querying using APIs (e.g., the query may include a patient identifier, a document identifier, a medical provider identifier, etc.; and the query may return the patient medical data), downloading the patient medical data (e.g., from the datastore, from a third-party service, or from any other source), and/or otherwise retrieved.

In a second variant, determining patient medical data may include receiving the patient's medical data from a user interface and/or any other suitable interface.

In some aspects, the patient medical data is normalized. Within patient medical data records, codes, such as industry standard codes, may be used to describe the data. Once the data is received, the codes within the data from different sources may be normalized such that the codes are consistent across the data records.

However, the patient medical data may be otherwise received.

5.2 S200—Extracting Attributes and Observed Properties from the Patient Medical Data.

In S200, attributes (e.g., “entities”) and observed properties may be extracted from the patient medical data to update a profile for the patient (e.g., in S400). In some aspects, once the patient medical data is received and normalized, the data may be compared (e.g., matched) to a set of predetermined attributes and a set of predetermined properties to identify which attributes and properties exist within the data. In general, attributes correspond to actions and entities of particular relevance to the patient (such as a conclusion, characteristic, measurement, treatment, or procedure), while properties correspond to specific details of an attribute. In some aspects, the predetermined attributes include industry standard terms. The attributes may include, for example: diagnosis (e.g., primary diagnosis, secondary diagnosis), comorbidity, adverse event, medication, lab result, observation panel (e.g., gene observation panel, etc.), biomarker, diagnostic procedure, therapeutic procedure, treatment group, immunizations, pathology-specific datatypes (e.g., TNM stage, tumor location, sage, grade, tumor feature, tumor status, molecular type, histologic type, tumor size, performance status, etc.), and/or any other suitable attribute.

Each attribute may be associated with a set of properties (observed or inferred). The observed properties may be extracted from the patient medical data. The observed properties may be: text strings, numerical values, data values, and/or be another datatype. The observed properties may be used to determine the inferred properties, used to update the profile (e.g., in S400), and/or otherwise used. Examples of observed properties include: date, date range, medical code, related ontology codes, relationships to other attributes, ingredients, dosage, route (e.g., ingestion, injected, etc.), form (e.g., syrup, pill, injection, etc.), panel name, test company, specimen source, test method, sample collection date, lab test, result value or range, result units, and/or any other suitable property that may be determined from patient medical data. For example, a given diagnosis may be an attribute, while the date of the diagnosis and the medical code for the diagnosis may be observed properties of that diagnosis attribute. Similarly, a particular medication may be an attribute, while the start date, the ingredients, the dosage, the route, and the form may be observed properties of that medication attribute. In another example, a particular lab result may be an attribute, while the lab panel name, test company, specimen source, test method, sample collection date, type of test, result value or range, and result units may be observed properties of that lab result attribute. The predetermined properties may or may not correspond to standard medical terms (e.g., some properties may be terms, while other properties may be patient-specific values).

Those attributes from the received patient medical record that match predetermined attributes are identified as extracted attributes. For example, an extracted attribute may be a predetermined attribute that matches a first data from the data set.

Those properties from the received patient medical record that match predetermined properties are identified as observed properties. For example, an observed property may be a predetermined property that matches a second data from the data set. Each extracted attribute in the set of extracted attributes may be related to a subset of observed properties in the set of observed properties for the patient.

In a first variant, extracting attributes and observed properties may include: parsing the patient medical data into a standardized medical terminology via a parser module or the like (e.g., parsing the data within the data set into a set of medical terms via machine learning or natural language processing); matching the medical terms (in the medical terminology) to predetermined attributes and predetermined properties in the datastore; and adding matched attributes and matched properties to a patient specific set of extracted attributes and observed properties. Matching the medical terms and predetermined properties may be performed by matching values, using regular expressions, and/or using any other suitable language processing technique.

A specific example of parsing text is described in U.S. application Ser. No. 16/432,592 filed 5 Jun. 2019, which is incorporated in its entirety by this reference. However, the patient medical data may be otherwise parsed.

In a second variant, extracting attributes and observed properties may include: determining word embeddings from the patient medical data; and using the word embeddings to compare against at least one of the set of predetermined attributes and at least one of the set of predetermined properties to determine a patient specific set of extracted attributes and set of observed properties. Such a comparison may involve fuzzy matching.

A specific example of using word embeddings to generate extracted attributes and observed properties is described in U.S. application Ser. No. 16/565,250 filed on 9 Sep. 2019, which is incorporated in its entirety by this reference.

In a third variant, extracting attributes and observed properties may include: extracting statements from the patient medical data using a model trained to extract statements associated with a given attribute type, and adding the statements to a patient specific set of extracted attributes and set of observed properties. The model may extract observed properties (associated with the respective attribute type) from the medical data as part of the statement, or separately extract the observed properties.

In a fourth variant, extracting attributes and observed properties may include: extracting statements from the patient medical data using a learning model trained to identify medical terms, classifying each extracted statement as one of a predetermined set of attribute types, and adding the classified statement (the “attribute”) to a patient specific set of extracted attributes.

However, the attributes and observed properties may be otherwise extracted from the patient medical data.

Extracting the attributes and observed properties may include recording provenance (e.g., the particular patient medical data identifier; type of patient medical data; where the attribute and/or observed property was found in the patient medical data, such as page, paragraph, line, etc.; the raw text; and/or any other data); storing the provenance in the datastore, and/or otherwise determining the provenance.

However, the extracted attributes and observed properties may be otherwise determined.

### 5.3 S300—Determining Inferred Properties for the Extracted Attributes.

In S300, inferred properties for the extracted attributes are determined. An inferred property is a property of an attribute that is not explicitly present (i.e., observed) in the patient's medical record. The inferred properties may be used for updating the profile for the patient (e.g., in S400). The inferred properties may be determined based on the observed properties, based on provenance associated with an extracted attribute and/or observed property, and/or any other suitable information. The inferred properties may include: ontology codes (e.g., custom, predetermined codes stored in the datastore, wherein each code may represent a curated collection of one or more medical codes); terminology codes (e.g., code, such as HGNC:34613; medical name, such as TRE-TTC13-1; terminology source, such as HGNC; etc.); name codes (e.g., medical name, such as TTRE-TTC13-1; terminology source, such as HGNC; etc.); other identifiers; and/or other inferred properties. One inferred property type may optionally be associated with values for other inferred property types.

For example, an ontology code may be associated with one or more terminology codes. It should be noted that terminology codes (also referred to herein as medical codes) are well established by the medical community within the industry. An ontology may be a curated list of terminology codes that share some commonality, such as being related to the same illness, being related to the same type of attribute, etc. For example, a lung cancer ontology may include a curated list of standard medical codes that are related to lung cancer, such as medical codes for cancer treatments, medications, diagnoses, and/or symptoms. The ontology itself may be identified by an ontology code. The ontology may be curated by an internal team at a given organization, and different organizations may have different ontology definitions and ontology codes. The ontology may additionally include an identification of relationships between certain terminology codes in the ontology. Some terminology codes may be included in more than one ontology. In some aspects, a set of one or more ontologies that map to a given extracted attribute (e.g., based on the terminology codes associated with the extracted attribute) are identified. Identifying the set of ontologies may include determining a standardized code, such as the terminology code, corresponding to the extracted attribute. Once the standardized code has been determined, the code may be sent to an ontology lookup service. In some aspects, the ontology lookup service returns all ontologies in a data store associated with the ontology lookup service that contain, or are otherwise tagged as related to, the standardized code. The corresponding ontology codes, as well as the information contained within the ontologies, may added to the set of inferred properties for the extracted attribute.

Each attribute may have one or more types of inferred properties, and one or more instances of each inferred property type. For example, an attribute may be assigned an ontology code, wherein the attribute is further associated with all parent and child ontology codes within the assigned ontology code's ontology branch. In another example, an attribute may have a single terminology code.

In some aspects, the inferred property value is specific to the attribute's attribute type, but may alternatively be generic. For example, the diagnosis attribute type may have a diagnosis-specific ontology code set and a diagnosis-specific terminology code set, while the medication attribute type may have a different medication-specific ontology code set and a different medication-specific terminology code set.

The inferred property for each attribute may be determined using similar methods as discussed above for S200, or be otherwise determined.

For example, the inferred property (e.g., code) for an attribute may be determined by comparing the attribute's word embeddings against the predetermined word embeddings associated with the inferred property.

In a second example, the inferred property for an attribute may be determined by comparing the attribute's values (e.g., text string) against the predetermined set of values associated with the inferred property. However, the inferred property may be otherwise determined.

In a third example, determining inferred properties for an extracted attribute may include mapping the medical code to an ontology code of the set of ontology codes. The medical code may be mapped to an ontology code: using a lookup table, by querying a datastore (e.g., the query may include the extracted attribute and/or the observed properties and the query may return the ontology code), and/or otherwise mapped to the ontology code.

In an illustrative example, if the attribute includes “Dexamethasone,” then the inferred properties may include the ontological codes for “dexamethasone” specifically, dexamethasone's drug class “glucocorticoid,” the higher-level drug class “steroid,” and optionally ontological codes for specific brands of dexamethasone (e.g., Ozurdex™, Maxidex™, DexPak 6 Day™, DexPak 10 Day™, LoCort™, etc.).

However, the inferred properties may be otherwise determined.

### 5.4 S400—Updating a Profile for the Patient.

In S400, a profile for the patient is updated. S400 may function to merge extracted attributes, observed properties, and/or inferred properties from multiple different sources and optionally multiple different providers into the profile for the patient. The profile may be updated when new medical data is received for a patient, when new attributes are available for a patient, at a predetermined interval, and/or at any other suitable time.

The merging may be performed using a set of merging rules that are associated with the set of ontologies, the set of extracted attributes, the set of observed properties, and the set of inferred properties that have all been identified. The merging further includes merging into a set of database records, which corresponds to a profile of the patient whose medical data was first received.

Additionally, the merging may include determining whether multiple attributes in the set of extracted attributes correspond to different versions of a same information. The merging may also include consolidating the multiple attributes to a single attribute within the set of extracted attributes. Within the multiple attributes, a first attribute may have a first observed property, a second attribute may have a second observed property, and so on such that the single attribute includes both the first and second observed property. The first observed property may be the start date, for example for starting a medicine, and the second observed property may be the end date, for example for ending a medicine.

Such merging and consolidating may be performed for each group of multiple attributes in the set of extracted attributes. By repeating and consolidating, a set of single attributes may be generated within the set of extracted attributes. Each single attribute within the set of extracted attributes may be associated with at least one date, for example a start or end date. Once the single attribute is associated with at least one date, the merging can further include displaying each of the single attributes within the set of extracted attributes along a timeline based on the associated date. The display may be display in a user interface generated from the profile of the patient.

The database records, which correspond to extracted attributes, observed properties, and inferred properties, within the patient profile may be linked within a database based on attribute and property relationships contained in the set of ontologies. Once the database records have been linked, the extracted attributes may also be further verified based on the set of observed properties and set of inferred properties.

Merging may also include displaying, within the user interface generated from the profile of the patient, each extracted attribute together with its subset of observed properties.

The profile may include: all (or a subset thereof) extracted or existing attributes and observed and/or inferred properties per attribute. The profile may be a data entry in a datastore, a physical profile (e.g., printable), and/or otherwise represented. The profile may be presented on a user interface, provided via download, provided as a response to a query for the profile, and/or otherwise provided.

The extracted attributes and observed properties may be added to the patient profile when the extracted attributes and observed properties have more information and/or more updated information than the existing attributes and properties. For example, the profile may be added to when: extracted attributes and properties of the attribute do not match the existing attributes and existing properties for the patient (e.g., existing attributes and existing properties that are part of the patient's profile); extracted attributes matching existing attributes but extracted properties not matching existing properties; extracted attributes match existing attributes and existing properties do not include the observed properties; and/or performed at any other suitable time. Additionally or alternatively, when the patient is not associated with a profile, S400 may include: creating a new profile using the extracted attributes, observed properties, and inferred properties; and storing the new profile in the datastore.

S400 may include: matching extracted attributes with existing attributes and merging the extracted attributes (e.g., from the patient specific set of extracted attributes) with existing attributes (e.g., attributes that are already part of the profile); verifying the attributes and properties associated with the attributes; and/or any other suitable elements. In some aspects, merging extracted attributes and verifying the attributes are performed in parallel, but may additionally or alternatively be performed in series (e.g., verifying may be performed after merging the attributes).

In some aspect, attributes may be matched for deduplication and/or merging based on the attribute type (e.g., “diagnosis,” “medication,” “procedure,” etc.); the inferred properties associated with the attributes (e.g., same diagnosis type, same medication type, same procedure type, etc.), the observed properties associated with the attributes; the provenance; and/or any other suitable information.

In a first variation, each attribute type may be associated with a set of matching rules, which are used to determine if the extracted attribute matches an existing attribute. The rules may compare the inferred parameters of the extracted and existing attributes (e.g., the ontology codes, the terminology codes, etc.), the observed and existing values of a rule-specified parameter set (e.g., start date, end date, occurrence date or event date, ingredients, route, form, test company, panel name, etc.), or other parameters to determine whether the extracted and existing attributes match. The rules may require an exact match, an approximate match (e.g., dates within a threshold range of each other; overlapping dates; fuzzy matches; etc.), and/or other match type.

In this variation, S400 may include: for each extracted attribute, determining the existing attributes sharing the same attribute type as the extracted attribute (e.g., diagnosis, medication, etc.); identifying existing attributes matching (e.g., exact or related match) the same inferred parameter (e.g., ontology code, otherwise terminology code; otherwise name code; etc.) as the extracted attribute; identifying the rule associated with the attribute type; identifying the observed and existing parameters, specified by the rule, for the extracted and existing attributes; and evaluating whether the extracted and existing attributes are a match based on the rule (example shown in FIG. 3). When the extracted and existing attributes are considered a match, then the observed parameters from the extracted attribute may be merged into the patient profile for the existing attribute. When the extracted and existing attributes are not considered a match, then a conflicts resolution process may be initiated (e.g., a second set of logic is applied to resolve the conflicting information, the conflicting information is sent to a user for validation, etc.). However, the extracted attributes may be otherwise merged with the patient profile.

In one example, after the extracted attribute is matched to an existing attribute, the rule associated with the attributes' type specifies that the matching attributes may be further matched based on dates. In a first example, matching attributes may be merged (e.g., all but one of the matching attributes may be filtered out or otherwise removed; the data between the matched attributes are consolidated into a single instance; etc.) when each attribute of the match is associated with the same date (e.g., diagnostic procedure date, immunization date, etc.). In a second example, matching attributes may be merged into a single attribute with a new date range property that includes a start date of a first attribute of the match and an end date of a second attribute of the match. In a third example, matching attributes may be merged based on date ranges (e.g., adjusting date ranges when a matching attribute is associated with an earlier or later date than that of an existing date range). Additionally or alternatively, merging the matching attributes may be performed based on ontology codes, medical codes, ingredients, dose, and/or any other properties.

In a first specific example of merging matching attributes, diagnostic procedure attributes (e.g., X-Rays, CT scan, MRI, etc.) may be merged if matching attributes include properties specifying the same date of the procedure, the same medical code, the same ontology code, and/or any other same property.

In a second specific example of merging matching attributes, therapeutic procedure attributes (e.g., chemotherapy, radiation, etc.) may be merged and properties may be updated (e.g., number of occurrences of the therapeutic procedure, date range, etc.).

In a third specific example of merging matching attributes, medications and/or treatment groups may be merged based on ingredients, start date, end date, form, dose, and/or any other suitable property.

In a second variation, S400 may include: adding the extracted attributes to the existing attributes; and filtering the set of all attributes (e.g., extracted and existing attributes) to determine a set of merged attributes. Filtering the set of all attributes may include determining two or more matching attributes and merging the matching attributes and/or properties of the matching attributes.

The second variation may optionally include de-duplicating attributes based on extracted and/or existing observed and/or inferred properties. De-duplicating attributes may include removing all but one matching attribute and associated properties from the set of all attributes.

In a third variation, S400 may include matching attributes based on a generic set of rules, and merging observed parameter values according to a set of rules (e.g., generic set of rules or attribute-specific set of rules).

However, the attributes may be otherwise matched.

S400 may also include verifying the attributes and properties associated with the attributes may include determining two or more conflicted attributes and verifying conflicted attributes.

Determining when two or more attributes conflict may be performed based on observed and/or inferred properties associated with attributes. In a first example, a first attribute may be in conflict with a second different attribute when the first and second attributes are of the same attribute type, and when one or more properties match (e.g., same date, same date range, same ingredients, same dose, etc.).

In the first variant, verifying conflicted attributes may be performed based on the inferred properties, observed properties, provenance, patient supplied information, and/or be otherwise based. In a first embodiment, conflicted attributes may be corrected using the associated ontology codes of the conflicted attributes (e.g., when a first attribute and a second attribute are associated with the same ontology code and different medical codes and the second attribute's date is after the first attribute's date, then the second attribute may replace the first attribute as the corrected version of the first attribute). In a second embodiment, conflicted attributes may be corrected based on the attribute with the most recent date and/or most recent date range. In a third embodiment, conflicted attributes may be corrected based on a voting mechanism (e.g., when multiple attributes of a same type match and one attribute of the type does not, majority vote may be used to correct the conflicted attribute, wherein correcting the conflicted attribute may include removing the attribute from the set of all attributes, or otherwise resolving the conflict). In a fourth embodiment, conflicted attributes may be corrected using provenance associated with the existing and/or extracted attributes (e.g., using medical provider notes, such as a note specifying a corrected attribute and/or a corrected property of an attribute; any other provenance). In a fifth embodiment, conflicted attributes may be corrected manually (e.g., by presenting one or more attributes and properties to the user and receiving corrected attributes and corrected properties from the user). However, conflicted attributes may be otherwise verified.

S400 may optionally include creating a profile, wherein extracted attributes and associated properties (observed and/or inferred) may be merged and/or verified using the processes described in the first variant.

However, the profile may be otherwise determined. Additionally, the process described herein may be performed for more than one patient.

FIG. 3 describes an implementation of the method 300 according to an aspect of the present invention. A medical record 310 is received by an attribute module 312. The attribute module 312 may include a processing device that executes one or more operations of S200 to obtain one or more attributes and/or observed properties, as described above with respect to FIG. 1. The attribute module 312 generates a statement 314. The statement 314 may include at least one attribute having an attribute type (also referred to as an entity and entity type, respectively) derived from the medical record 310. While the medical record 310 may have multiple attributes and/or observed properties identified by the attribute module 312, this example will refer to only one attribute for ease of explanation. The statement 314 may include an identification of the attribute type (in this example, “stmt.entitytype1”. which refers to a statement of entity type1), along with any observed properties (also referred to herein as parameters) corresponding to the attribute (in this example, “dates”). The statement 314 containing the attributes, attribute types, and/or observed properties may then be input into an inferred property module 316. A statement may refer to informational concepts that are broken down from, for example, medical records. A statement may be, for example, a sentence in a medical record. Statements, along with their properties that may be used in code or rule matching, will be described with respect to FIG. 4. In some aspects, the attribute is processed and/or normalized so that it is represented as or otherwise includes a standard medical code corresponding to that attribute.

The inferred property module 316 may then identify one or more inferred properties corresponding to statement 314 based on the extracted attributes, attribute (also referred to herein as entity) types, and/or observed properties. The inferred property module 316 may include a processing device (which may be the same or different processing device as attribute module 312) that executes one or more operations of S300 to determine one or more inferred properties, as described above with respect to FIG. 1. The inferred property module 316 may output a record 318 containing information about statement 314, including the entity type (“stmt.entitytype1”), observed properties (also referred to herein as parameters) of the entity type (e.g., “dates”), and a code associated with the attribute. In the example of FIG. 3, the code associated with the attribute is a standard medical code (“stmt.code1”) corresponding to the extracted attribute. Inferred properties may also be included as parameters in record 318, or may be added as metadata tagged to record 318.

In some aspects, the inferred property module 316 may infer one or more ontology codes that correspond to the attribute. An ontology may be a curated list of standard medical codes that share some commonality, such as being related to the same illness, being of the same type of attribute, etc. For example, a lung cancer ontology code may include a curated list of standard medical codes that are related to lung cancer, such as medical codes for cancer treatments, medications, diagnoses, and/or symptoms. The ontology itself may be identified by an ontology code. In some aspects, the ontology code for a given attribute is determined using an ontology lookup service. In some aspects, the ontology lookup service identifies one or more ontologies containing a medical code corresponding to the attribute, and returns one or more ontology codes corresponding respectively to the one or more ontologies.

In some aspects, a given attribute (as identified by its standard medical code) may be associated with one or more ontologies, in that the attribute is included in the curated list of medical codes for that ontology. For example, a lung cancer diagnosis attribute may correspond to a particular medical code. That particular medical code may be included in a curated set of codes for a lung cancer ontology. The lung cancer ontology may be associated with a given lung cancer ontology code. That lung cancer ontology code may be identified by inferred property module 316 as an inferred property of the lung cancer diagnosis attribute. Any given attribute may be included in multiple ontologies, only one ontology, or no ontologies depending on the nature of the attribute.

A related code database 320 may be consulted to determine other medical codes (e.g., “related code2”) that are related to the given attribute. For example, the other medical codes may be those included in the ontology identified by the inferred ontology code. The other medical codes may be considered as “related” to the attribute because they are part of the same ontology, and thus added to the record for the attribute. These related codes constitute inferred properties of the attribute. Using the information from related code database 320, an updated record 322 is generated that contains, for a given statement (“stmt”), the extracted attribute type (“entitytype1”), codes corresponding to the attribute (“code1” and “related code2”), and any parameters (“dates, . . . ”). Parameters may include observed and/or inferred properties corresponding to the attribute extracted from statement 314.

Record 322 may be incorporated into a patient profile via an entity matching process and a code matching process.

To begin the entity matching process, the entity type (e.g., “entitytype1”) from record 322 is compared to a patient profile 332 in a matching step 330. In the matching step 330, it is determined whether or not the patient profile 332 already contains information corresponding to the information from statement 314 with the same entity type (“entitytype1”). If information matching the same entity type already exists in patient profile 332, then the method proceeds in step 334 to identify, in an entity listing 336, existing entities (“entity3,” “entity4”) in the patient profile 332 that correspond to the same entity type (“entitytype1”). Codes (“code3,” “code2”) and properties (“parameter set 3,” “parameter set 4”) that respectively correspond to the existing entities are also identified from patient profile 332. If a matching entity type does not already exist in patient profile 332, then the method proceeds in 336 to add the entity (“entityn”) from statement 314 to a new entity listing 336 for the entity type, along with its respective code (“coden”) and properties (“parameter set n”).

To begin the code matching process, the codes identified in record 322 are compared to the codes identified in entity listing 336 in a matching step 340. In the matching step 340, it is determined whether or not the codes identified in record 322 are compatible as any of the codes identified in entity listing 336. If it is determined in step 340 that no code exists that is compatible in the entity listing generated from patient profile 332, then the method proceeds in step 344 to add the entity (“entityn”) from statement 314 to the entity listing 336 for that entity type (“entitytype1”), along with its respective code(s) (“coden”) and properties (“parameter set n”).

However, if it is determined in matching step 340 that a compatible code already exists, then the corresponding entry from entity listing 336 is extracted in step 342 as existing entry 356. For this example, record 322 shows that statement 314 contains information corresponding to “code2.” It is determined in matching step 340 that “code2” is already present in patient profile 332 for the same entity type, because of its inclusion in entity listing 334. Accordingly, in step 342, the entry containing “entity4,” “code2,” and “parameter set 4” is extracted from entity listing 336 as existing entry 356.

Once existing entry 356 is extracted, then the properties from existing entry 356 (“parameter set 4”) and the properties from record 322 (“dates, . . . ”) containing the same code are input in step 352 to a rules engine 350. Rules engine 350 identifies the entity type corresponding to existing entry 356 and record 322 (here, “entitytype1”). Rules engine 350 uses rules specific to that entity type in order to merge the new information from record 322 into existing entry 356. For example, information having a diagnosis entity type may be merged differently from information having a lab test entity type, and thus would have different matching rules. Any conflicts between the information in record 322 and the information in existing entry 356 are resolved according to the entity-specific matching rules, and the correct information is merged into existing entry 356 in step 360, thus updating the patient profile 332. The rules engine 350 may include a processing device that executes one or more operations of S400 to merge information into and update the patient profile, as described above with respect to FIG. 1.

While FIG. 3 illustrates an aspect where each incoming entity is compared to the patient profile in step 330, entities and/or statements may instead be aggregated without reference to an existing patient profile. In such an aspect, multiple statements for a given entity may be compared to each other and aggregated as appropriate, at which point the patient profile is updated. Such a comparison treats all statements as equal for consideration during the aggregation, so that there is no bias towards statements that may already have existed in the patient profile. Further, this comparison between original statements, rather than a prior aggregation or summary as contained in the patient profile, allows for comparison between complete ground truths from the statements such that no relevant information is missing during the aggregation. Additionally, such a comparison allows aggregation schemes and the aggregated data in the patient profile to evolve and/or be overridden instead of relying on an aggregation scheme as may have been previously used (e.g., the patient profile previously included aggregated data X, but new data requires a distinction between X′ and X″ that was not previously accounted for, or where X was proven false based on new data). Such a comparison also allows the method to scale, even as the patient profile itself grows. Finally, such a comparison allows processes for multiple entities and/or multiple patients to be run in parallel, with the portions of the patient profile corresponding to those entities being updated as aggregation is completed.

FIG. 4 is a process flow diagram for a method 400, according to an aspect of the present invention. The method 400 illustrates how a statement is processed and merged with aggregated data. The method 400 expands upon the method 100 described in FIG. 1. To begin method 400, a medical record 402 is received by a statement generator 404. The statement generator 404 processes the medical record 402 to extract a plurality of statement records 406. Each statement record in the plurality of statement records 406 includes an extracted statement from medical record 402 along with an associated code and parameter set. In the example of FIG. 4, an example statement record 406 includes “statement1,” “code1,” and “parameter set1.” These extracted statement records 406 may then be input into a statement service 408. The statement service 408 stores the plurality of statement records 406 in a statement store 410 (e.g., a database).

The statement store 410 stores all statements from the medical record 402. Storage of the original statements in the statement store allows the original truth statement to be maintained, even if some detail does not convey into the final patient profile due to the aggregation process. Once all statements related to a particular entity (e.g., medical code) from the medical record 402 have been sent to the statement store 410, as determined by the statement generator 404, statement generator 404 may send a message, such as message 416, for entry into a queue 412. The message, such as each of messages 416-418, identifies content to be aggregated by aggregate service pod 460. From the queue 412, a content based router 414 distributes the message(s) to an available processing engine in aggregate service pod 460. For example, the content based router 414 may send message 416 to a first instantiation of the aggregate service pod 460, message 417 to a second instantiation of the aggregate service pod 460, and message 418 to a third instantiation of the aggregate service pod 460. While FIG. 4 illustrates only three messages and three instantiations, a person of skill in the art will recognize that the number of messages able to be handled by aggregate service pod 460 at any given time depends on the number of processes able to be executed in parallel by aggregate service pod 460 (e.g., based on the number of processor cores and/or number of threads able to be processed at a given time by a given processor core).

Each message sent from the statement generator 406 to queue 112 may include information used by the aggregate service pod 460 to request relevant statements from the statement service 408 for aggregation. For example, the messages may include data and/or metadata related to a set of statements to be considered for aggregation by the aggregate service pod 460. Such statement data and/or metadata may include, for example, the patient ID, any related code and/or attribute value, and any related parameters. The code identified in the patient-specific message can be, for example, a medical code or an ontology code. As illustrated in FIG. 4, patient-specific message 416 includes message1, patient id1, code1, and parameters1. Patient-specific message 417 includes message2, patient id2, code2, and parameters2. Patient-specific message 418 includes message3, patient id3, code3, and parameters3. The messages 416-418 may be input into different aggregation processes of aggregate service pod 460.

Once a message is received by the aggregate service pod 460, the message is analyzed to identify information needed to request statements from the statement service 408. In the example shown in FIG. 4, the aggregate service pod 460 extracts the patient ID (“PatientId 1”) and code (“code 1”) from the input message 416, and sends a request for statements corresponding to that patient ID and code to the statement service 408. In step 470, the statement service 408 retrieves the plurality of statement records 406 corresponding to that patient ID and code from the statement store 410, and sends those retrieved statement records to aggregate service pod 460. In some aspects, the code can be either an ontology code, if present, or a medical terminology code, if the ontology code is absent. Additionally, in some cases of generic aggregation, for example when the ontology code is not present, the terminology code may also be used for aggregation. In some aspects, the request for statements sent by the aggregate service pod 460 to the statement service 408 may not include the code, depending on, for example, the situation or entity type rules. As a result, the statement service 408 may return statements having multiple different codes. Those multiple codes are later checked for compatibility, as will be described below with respect to step 430.

In some aspects, aggregate service pod 460 receives the statement records returned by the statement service 408 in response to its request (i.e., those statements that satisfied the parameters provided, which in the example of FIG. 4 include “PatientId 1” and “code 1”), and separates the returned statement records into individual statement records 420, 422, 424, and 426 based on, for example, their statement IDs. For example, statement 420 has statement ID “Statement 1,” statement 422 has statement ID “Statement 2,” statement 424 has statement ID “Statement 3,” and statement 426 has statement ID “Statement 4.” Each individual statement record 420, 422, 424, and 426 contains a given statement extracted from medical record 402, along with a corresponding code and any observed or inferred properties associated therewith. For example, individual statement record 420 (“statement1”) includes “code1” and “parameters1.” Individual statement records 422, 424, and 426 share the same code (“code1”), but have different statements IDs (“Statement 2,” “Statement 3,” “Statement 4”) and properties (“parameter set 2,” “parameter set 3,” “parameter set N”). As individual statement records 420, 422, 424, and 426 were all returned in response to a request for statements associated with a particular patient ID and code, that patient ID and code (which may be an identical or compatible code) will typically be shared across each of the returned individual statement records 420, 422, 424, and 426. However, some returned statements may be missing one or more of these values, such as if the statement was associated with a patient ID but no particular code (e.g., if the code field for the statement is empty), or if the code associated with the statement contains modifiers to a same base code. In some aspects, if no code was included in the request from the aggregate service pod 460 to the statement service 408, the returned statements may be associated with a variety of codes.

The individual statement records are then evaluated for inclusion into the patient profile for that patient by aggregate service pod 460. In step 430, the medical code(s) or ontology code(s) from the individual statement records (such as records 420, 422, 424, and 426), which may include the medical code for the attribute and any related medical codes, are compared to each other to determine whether the codes are compatible. Typically the codes will be compatible if statement service 408 has been requested to return statements specifically containing that code, but sometimes there will be an outlier statement with no code, as mentioned above. In some aspects, if no code was included in the request from the aggregate service pod 460 to the statement service 408, the returned statements may be associated with a variety of codes which then need to be checked for compatibility. In some aspects, an equality check may be performed to determine whether two codes are compatible. In some aspects, there may be different logic to determine whether certain codes are compatible, as would be evident to a person of skill in the art. If the code from a given individual statement record is not compatible with the code from another individual statement record, then any attributes (also referred to as entities) 434 associated with the incompatible statement record are extracted and handled as independent entities 434.

If the codes from the individual statement records are compatible with each other, then aggregate service pod 460 must determine whether any of the individual statement records having compatible codes for a particular attribute/entity should be merged with each other. Accordingly, in step 432, it is determined whether any compatibility rules specific to that attribute type are applicable. The compatibility rules check whether specific attribute type parameters from the statements match with each other. If no attribute type-specific compatibility rules match for a particular attribute, then that attribute is handled as an independent entity 434. If it is determined in step 432 that compatibility rules match for that attribute type, then the information from individual statement records corresponding to that attribute type is merged in step 436 produce a merged attribute 438. As statements 420, 422, 424, and 426 may contain different entity types, step 432 (along with step 430) may be performed for each entity type to produce a set of merged attributes 438.

For example, if the code is an ontology code for “pancreatic cancer,” attributes can include a treatment, a drug, etc. Each of those attributes may be associated with multiple statements containing different parameters (e.g., start date, end date, lab name, etc.). If there are particular compatibility rules for a given treatment attribute, for example, statements corresponding to the given treatment attribute under the “pancreatic cancer” ontology code will be processed according to step 436 to produce a merged entity 438. If the compatibility rules do not match, entity 434 is produced.

In step 440, attributes 434 and/or merged attributes 438 are analyzed to determine whether that same attribute (entity) with the same statement ID already exists in a patient profile corresponding to the patient ID. If an attribute with that statement ID exists, then in step 442, an existing record in the patient profile for that attribute is updated. Each attribute is associated with one or more statement IDs, maintaining the provenance of the record for that attribute. In some aspects, when it is determined that an attribute with that entity ID already exists in the patient profile, it is determined whether the same statement ID referenced by that attribute also already exists in the patient profile. If so, the entity corresponding to that statement ID in the attribute-specific portion of the patient profile is simply updated. If not, a new entity corresponding to the statement ID is added in step 444 to the attribute-specific portion of the patient profile.

The attribute records from step 442 and/or step 444 constitute aggregated data, and are output as aggregated data 450 for that patient (e.g., “PatientId 1”). Each record includes the attribute along with its corresponding information, such as medical code, entity type, and properties (including observed and inferred properties). For example, the record within the aggregated data 450 for attribute “entity 1” includes its code (“code 1”), its entity type (“entity type 1”), and its set of properties (“params1”). The aggregated data 450 then is sent to an object store service 452. The object store service 452 stores the aggregated data 450 in entity store 454, which contains the newly created or newly updated patient profile 456. In some aspects, patient profile 456 organizes each entity by entity type. For example, as illustrated in FIG. 4, the newly created patient profile 456 may include a listing of entity types, such as entity type1, entity type2, . . . entity typeN. Each entity type1 includes entity 1 up to entityN.

Embodiments of the system and/or method may include every combination and permutation of the various system components and the various method processes, wherein one or more instances of the method and/or processes described herein may be performed asynchronously (e.g., sequentially), concurrently (e.g., in parallel), or in any other suitable order by and/or using one or more instances of the systems, elements, and/or entities described herein.

As a person skilled in the art will recognize from the previous detailed description and from the figures and claims, modifications and changes may be made to the preferred embodiments of the invention without departing from the scope of this invention defined in the following claims.

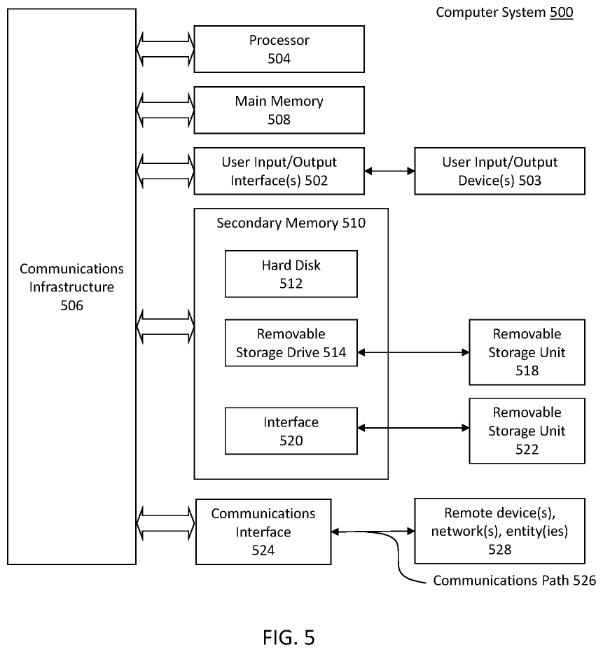

Various aspects may be implemented, for example, using one or more computer systems, such as computer system 500 shown in FIG. 5. Computer system 500 may be used, for example, to implement one or more aspects of method 100 of FIG. 1, method 300 of FIG. 3, and/or method 400 of FIG. 4. Computer system 500 may be any computer capable of performing the functions described herein. Computer system 500 may be any well-known computer capable of performing the functions described herein.

Computer system 500 includes one or more processors (also called central processing units, or CPUs), such as a processor 504. Processor 504 is connected to a communication infrastructure or bus 506.

One or more processors 504 may each be a graphics-processing unit (GPU). In an aspect, a GPU is a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 500 also includes user input/output device(s) 503, such as monitors, keyboards, pointing devices, etc., that communicate with communication infrastructure 506 through user input/output interface(s) 502.

Computer system 500 also includes a main or primary memory 508, such as random access memory (RAM). Main memory 508 may include one or more levels of cache. Main memory 508 has stored therein control logic (i.e., computer software) and/or data.

Computer system 500 may also include one or more secondary storage devices or memory 510. Secondary memory 510 may include, for example, a hard disk drive 512 and/or a removable storage device or drive 514. Removable storage drive 514 may be a floppy disk drive, a magnetic tape drive, a compact disk drive, an optical storage device, tape backup device, and/or any other storage device/drive.

Removable storage drive 514 may interact with a removable storage unit 518. Removable storage unit 518 includes a computer usable or readable storage device having stored thereon computer software (control logic) and/or data. Removable storage unit 518 may be a floppy disk, magnetic tape, compact disk, DVD, optical storage disk, and/any other computer data storage device. Removable storage drive 514 reads from and/or writes to removable storage unit 518 in a well-known manner.

According to an exemplary aspect, secondary memory 510 may include other means, instrumentalities or other approaches for allowing computer programs and/or other instructions and/or data to be accessed by computer system 500. Such means, instrumentalities or other approaches may include, for example, a removable storage unit 522 and an interface 520. Examples of the removable storage unit 522 and the interface 520 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 500 may further include a communication or network interface 524. Communication interface 524 enables computer system 500 to communicate and interact with any combination of remote devices, remote networks, remote entities, etc. (individually and collectively referenced by reference number 528). For example, communication interface 524 may allow computer system 500 to communicate with remote devices 528 over communications path 526, which may be wired, and/or wireless, and which may include any combination of LANs, WANs, the Internet, etc. Control logic and/or data may be transmitted to and from computer system 500 via communication path 526.

In an aspect, a tangible, non-transitory apparatus or article of manufacture comprising a tangible, non-transitory computer useable or readable medium having control logic (software) stored thereon is also referred to herein as a computer program product or program storage device. This includes, but is not limited to, computer system 500, main memory 508, secondary memory 510, and removable storage units 518 and 522, as well as tangible articles of manufacture embodying any combination of the foregoing. Such control logic, when executed by one or more data processing devices (such as computer system 500), causes such data processing devices to operate as described herein.

Based on the teachings contained in this disclosure, it will be apparent to persons skilled in the relevant art(s) how to make and use aspects of this disclosure using data processing devices, computer systems and/or computer architectures other than that shown in FIG. 5. In particular, aspects may operate with software, hardware, and/or operating system implementations other than those described herein.

### CONCLUSION

It is to be appreciated that the Detailed Description section, and not any other section, is intended to be used to interpret the claims. Other sections may set forth one or more but not all exemplary aspects as contemplated by the inventor(s), and thus, are not intended to limit this disclosure or the appended claims in any way.

While this disclosure describes exemplary aspects for exemplary fields and applications, it should be understood that the disclosure is not limited thereto. Other aspects and modifications thereto are possible, and are within the scope and spirit of this disclosure. Further, aspects (whether or not explicitly described herein) have significant utility to fields and applications beyond the examples described herein.

Aspects have been described herein with the aid of functional building blocks illustrating the implementation of specified functions and relationships thereof. The boundaries of these functional building blocks have been arbitrarily defined herein for the convenience of the description. Alternate boundaries may be defined as long as the specified functions and relationships (or equivalents thereof) are appropriately performed. Also, alternative aspects may perform functional blocks, steps, operations, methods, etc. using orderings different than those described herein.

References herein to “one aspect,” “an aspect,” “an example aspect,” or similar phrases, indicate that the aspect described may include a particular feature, structure, or characteristic, but every aspect may not necessarily include the particular feature, structure, or characteristic. Moreover, such phrases are not necessarily referring to the same aspect. Further, when a particular feature, structure, or characteristic is described in connection with an aspect, it would be within the knowledge of persons skilled in the relevant art(s) to incorporate such feature, structure, or characteristic into other aspects whether or not explicitly mentioned or described herein. Additionally, some aspects may be described using the expression “coupled” and “connected” along with their derivatives. These terms are not necessarily intended as synonyms for each other. For example, some aspects may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

The breadth and scope of this disclosure should not be limited by any of the above-described exemplary aspects, but should be defined only in accordance with the following claims and their equivalents.

We claim:

1. A method for aggregating data comprising:
receiving a data source relating to a patient, the data source containing a data set;
matching data in the data set to at least one of a set of predetermined attributes and a set of predetermined properties to produce a set of extracted attributes and a set of observed properties for the patient, wherein each extracted attribute is a predetermined attribute that matches first data in the data set, and each observed property is a predetermined property that matches second data in the data set;
for at least one attribute in the set of extracted attributes, identifying a set of ontologies that maps to the at least one attribute;
determining a set of respective inferred properties for the at least one attribute based on the set of ontologies;
merging, according to a set of merging rules associated with the set of ontologies, the set of extracted attributes, the set of observed properties, and the set of respective inferred properties into a set of database records corresponding to a profile of the patient; and
linking database records corresponding to a plurality of extracted attributes, observed properties, and inferred properties within the profile of the patient based on the set of ontologies.

2. The method of claim 1, further comprising:
verifying, based on the set of observed properties and the set of respective inferred properties, the set of extracted attributes.

3. The method of claim 1, wherein the merging comprises:
determining that multiple attributes in the set of extracted attributes correspond to different versions of a same information; and
consolidating the multiple attributes to a single attribute within the set of extracted attributes.

4. The method of claim 3, wherein a first attribute in the multiple attributes has a first observed property, a second attribute in the multiple attributes has a second observed property, and the single attribute has both the first observed property and the second observed property.

5. The method of claim 4, wherein the first observed property is a start date and the second observed property is an end date.

6. The method of claim 3, further comprising repeating the determining and consolidating for each group of multiple attributes in the set of extracted attributes to generate a set of single attributes within the set of extracted attributes.

7. The method of claim 6, wherein each single attribute within the set of extracted attributes is associated with at least one date, the merging further comprising:
displaying, in a user interface generated from the profile of the patient, each single attribute within the set of extracted attributes along a timeline based on its respective at least one date.

8. The method of claim 1, wherein each extracted attribute in the set of extracted attributes is related to a respective subset of observed properties in the set of observed properties for the patient.

9. The method of claim 8, wherein the merging comprises displaying, in a user interface generated from the profile of the patient, each extracted attribute together with its respective subset of observed properties.

10. The method of claim 1, wherein each extracted attribute in the set of extracted attributes is related to a respective subset of inferred properties in the set of inferred properties for the patient.

11. The method of claim 1, wherein the matching comprises:
parsing the data in the data set into a set of medical terms using natural language processing; and
matching each medical term in the set of medical terms to the at least one of the set of predetermined attributes and the set of predetermined properties.

12. The method of claim 1, wherein the matching comprises:
determining word embeddings from the data in the data set; and
comparing the word embeddings against the at least one of the set of predetermined attributes and the set of predetermined properties to produce the set of extracted attributes and the set of observed properties for the patient.

13. The method of claim 12, wherein the comparing comprises fuzzy matching.

14. The method of claim 1, wherein the matching comprises:
extracting statements from the data in the data set using a model trained to extract statements associated with a given attribute type; and
adding the extracted statements to at least one of the set of extracted attributes and the set of observed properties for the patient.

15. The method of claim 1, wherein the set of predetermined attributes comprises a set of predetermined attribute types, and wherein the matching comprises:
extracting statements from the data in the data set using a learning model trained to identify medical terms;
classifying each extracted statement as one of the predetermined set of attribute types; and
adding the classified statement as an attribute in the set of extracted attributes for the patient.

16. The method of claim 1, wherein the set of extracted attributes is an existing set of extracted attributes and the set of observed properties is an existing set of observed properties, the method further comprising:
receiving a new data source relating to the patient, the new data source containing a new data set;
matching the new data set to the set of predetermined attributes to produce a new set of extracted attributes and a new set of observed properties for the patient;
determining that a new attribute in the new set of extracted attributes is the same as an existing attribute in the existing set of extracted attributes; and
merging any new observed properties corresponding to the new attribute into the set of database records corresponding to the profile of the patient for the existing attribute.

17. The method of claim 1, wherein identifying a set of ontologies comprises:
determining a standardized code corresponding to the at least one attribute;
sending the standardized code to an ontology lookup service; and
returning all ontologies in a datastore associated with the ontology lookup service that are tagged as related to the standardized code.

18. The method of claim 1, further comprising:
repeating the receiving, matching, identifying, querying, and merging for at least one other patient.

19. A system for aggregating data comprising:
a processor; and
a memory having instructions stored thereon that, when executed, cause the processor to perform operations comprising:
receiving a data source relating to a patient, the data source containing a data set;
matching data in the data set to at least one of a set of predetermined attributes and a set of predetermined properties to produce a set of extracted attributes and a set of observed properties for the patient, wherein each extracted attribute is a predetermined attribute that matches first data in the data set, and each observed property is a predetermined property that matches second data in the data set;
for at least one attribute in the set of extracted attributes, identifying a set of ontologies that maps to the at least one attribute;
determining a set of respective inferred properties for the at least one attribute based on the set of ontologies;
merging, according to a set of merging rules associated with the set of ontologies, the set of extracted attributes, the set of observed properties, and the set of respective inferred properties into a set of database records corresponding to a profile of the patient; and
linking database records corresponding to a plurality of extracted attributes, observed properties, and inferred properties within the profile of the patient based on the set of ontologies.

20. The system of claim 19, the operations further comprising:
verifying, based on the set of observed properties and the set of respective inferred properties, the set of extracted attributes.

21. The system of claim 19, wherein the merging comprises:
determining that multiple attributes in the set of extracted attributes correspond to different versions of a same information; and
consolidating the multiple attributes to a single attribute within the set of extracted attributes.

22. The system of claim 21, wherein a first attribute in the multiple attributes has a first observed property, a second attribute in the multiple attributes has a second observed property, and the single attribute has both the first observed property and the second observed property.

23. The system of claim 22, wherein the first observed property is a start date and the second observed property is an end date.

24. The system of claim 21, the operations further comprising repeating the determining and consolidating for each group of multiple attributes in the set of extracted attributes to generate a set of single attributes within the set of extracted attributes.

25. The system of claim 24, wherein each single attribute within the set of extracted attributes is associated with at least one date, the merging further comprising:
displaying, in a user interface generated from the profile of the patient, each single attribute within the set of extracted attributes along a timeline based on its respective at least one date.

26. The system of claim 19, wherein each extracted attribute in the set of extracted attributes is related to a respective subset of observed properties in the set of observed properties for the patient.

27. The system of claim 26, wherein the merging comprises displaying, in a user interface generated from the profile of the patient, each extracted attribute together with its respective subset of observed properties.

28. The system of claim 19, wherein each extracted attribute in the set of extracted attributes is related to a respective subset of inferred properties in the set of inferred properties for the patient.

29. The system of claim 19, wherein the matching comprises:
parsing the data in the data set into a set of medical terms using natural language processing; and
matching each medical term in the set of medical terms to the at least one of the set of predetermined attributes and the set of predetermined properties.

30. The system of claim 19, wherein the matching comprises:
determining word embeddings from the data in the data set; and
comparing the word embeddings against the at least one of the set of predetermined attributes and the set of predetermined properties to produce the set of extracted attributes and the set of observed properties for the patient.

31. The system of claim 30, wherein the comparing comprises fuzzy matching.

32. The system of claim 19, wherein the matching comprises:
extracting statements from the data in the data set using a model trained to extract statements associated with a given attribute type; and
adding the extracted statements to at least one of the set of extracted attributes and the set of observed properties for the patient.

33. The system of claim 19, wherein the set of predetermined attributes comprises a set of predetermined attribute types, and wherein the matching comprises:
extracting statements from the data in the data set using a learning module trained to identify medical terms;
classifying each extracted statement as one of the predetermined set of attribute types; and
adding the classified statement as an attribute in the set of extracted attributes for the patient.

34. The system of claim 19, wherein the set of extracted attributes is an existing set of extracted attributes and the set of observed properties is an existing set of observed properties, the operations further comprising:
receiving a new data source relating to the patient, the new data source containing a new data set;
matching the new data set to the set of predetermined attributes to produce a new set of extracted attributes and a new set of observed properties for the patient;
determining that a new attribute in the new set of extracted attributes is the same as an existing attribute in the existing set of extracted attributes; and
merging any new observed properties corresponding to the new attribute into the set of database records corresponding to the profile of the patient for the existing attribute.

35. The system of claim 19, wherein identifying a set of ontologies comprises:
determining a standardized code corresponding to the at least one attribute;
sending the standardized code to an ontology lookup service; and
returning all ontologies in a datastore associated with the ontology lookup service that are tagged as related to the standardized code.

36. The system of claim 19, the operations further comprising:
repeating the receiving, matching, identifying, querying, and merging for at least one other patient.

37. A non-transitory computer readable storage medium having instructions stored thereon that, when executed, cause a computer system to perform operations comprising:
receiving a data source relating to a patient, the data source containing a data set;
matching data in the data set to at least one of a set of predetermined attributes and a set of predetermined properties to produce a set of extracted attributes and a set of observed properties for the patient, wherein each extracted attribute is a predetermined attribute that matches first data in the data set, and each observed property is a predetermined property that matches second data in the data set;
for at least one attribute in the set of extracted attributes, identifying a set of ontologies that maps to the at least one attribute;
determining a set of respective inferred properties for the at least one attribute based on the set of ontologies;
merging, according to a set of merging rules associated with the set of ontologies, the set of extracted attributes, the set of observed properties, and the set of respective inferred properties into a set of database records corresponding to a profile of the patient; and
linking database records corresponding to a plurality of extracted attributes, observed properties, and inferred properties within the profile of the patient based on the set of ontologies.

38. The non-transitory computer readable storage medium of claim 37, the operations further comprising:
verifying, based on the set of observed properties and the set of respective inferred properties, the set of extracted attributes.

39. The non-transitory computer readable storage medium of claim 37, wherein the merging comprises:
determining that multiple attributes in the set of extracted attributes correspond to different versions of a same information; and
consolidating the multiple attributes to a single attribute within the set of extracted attributes.

40. The non-transitory computer readable storage medium of claim 39, wherein a first attribute in the multiple attributes has a first observed property, a second attribute in the multiple attributes has a second observed property, and the single attribute has both the first observed property and the second observed property.

41. The non-transitory computer readable storage medium of claim 40, wherein the first observed property is a start date and the second observed property is an end date.

42. The non-transitory computer readable storage medium of claim 39, the operations further comprising repeating the determining and consolidating for each group of multiple attributes in the set of extracted attributes to generate a set of single attributes within the set of extracted attributes.

43. The non-transitory computer readable storage medium of claim 42, wherein each single attribute within the set of extracted attributes is associated with at least one date, the merging further comprising:
displaying, in a user interface generated from database profile of the patient, each single attribute within the set of extracted attributes along a timeline based on its respective at least one date.

44. The non-transitory computer readable storage medium of claim 37, wherein each extracted attribute in the set of extracted attributes is related to a respective subset of observed properties in the set of observed properties for the patient.

45. The non-transitory computer readable storage medium of claim 44, wherein the merging comprises displaying, in a user interface generated from the profile of the patient, each extracted attribute together with its respective subset of observed properties.

46. The non-transitory computer readable storage medium of claim 37, wherein each extracted attribute in the set of extracted attributes is related to a respective subset of inferred properties in the set of inferred properties for the patient.

47. The non-transitory computer readable storage medium of claim 37, wherein the matching comprises:
parsing the data in the data set into a set of medical terms using natural language processing; and
matching each medical term in the set of medical terms to the at least one of the set of predetermined attributes and the set of predetermined properties.

48. The non-transitory computer readable storage medium of claim 37, wherein the matching comprises:
determining word embeddings from the data in the data set; and
comparing the word embeddings against the at least one of the set of predetermined attributes and the set of predetermined properties to produce the set of extracted attributes and the set of observed properties for the patient.

49. The non-transitory computer readable storage medium of claim 48, wherein the comparing comprises fuzzy matching.

50. The non-transitory computer readable storage medium of claim 37, wherein the matching comprises:
extracting statements from the data in the data set using a model trained to extract statements associated with a given attribute type; and
adding the extracted statements to at least one of the set of extracted attributes and the set of observed properties for the patient.

51. The non-transitory computer readable storage medium of claim 37, wherein the set of predetermined attributes comprises a set of predetermined attribute types, and wherein the matching comprises:
extracting statements from the data in the data set using a learning model trained to identify medical terms;
classifying each extracted statement as one of the predetermined set of attribute types; and
adding the classified statement as an attribute in the set of extracted attributes for the patient.

52. The non-transitory computer readable storage medium of claim 37, wherein the set of extracted attributes is an existing set of extracted attributes and the set of observed properties is an existing set of observed properties, the operations further comprising:
receiving a new data source relating to the patient, the new data source containing a new data set;
matching the new data set to the set of predetermined attributes to produce a new set of extracted attributes and a new set of observed properties for the patient;
determining that a new attribute in the new set of extracted attributes is the same as an existing attribute in the existing set of extracted attributes; and
merging any new observed properties corresponding to the new attribute into the set of database records corresponding to the profile of the patient for the existing attribute.

53. The non-transitory computer readable storage medium of claim 37, wherein identifying a set of ontologies comprises:
determining a standardized code corresponding to the at least one attribute;
sending the standardized code to an ontology lookup service; and
returning all ontologies in a datastore associated with the ontology lookup service that are tagged as related to the standardized code.

54. The non-transitory computer readable storage medium of claim 37, the operations further comprising:
repeating the receiving, matching, identifying, querying, and merging for at least one other patient.
